(19) United States
(12) Patent Application Publication
(10) Pub. No.: US 2007/0021361 A1
(43) Pub. Date: Jan. 25, 2007

(54) Methods and materials for modulating trpm2

(76) Inventors: Samuel J. Shuster, Edina, MN (US); Ulf N. G. Arvidsson, Lidingo (SE); Laura S. Stone, St. Louis Park, MN (US); Hong-Yan Zhang, Sundbyberg (SE); Lucy Vulchanova Hart, St. Paul, MN (US)

Correspondence Address: FISH & RICHARDSON P.C., PO BOX 1022, MINNEAPOLIS, MN, 55440-1022, US

(21) Appl. No.: 10/537,741

(22) PCT Filed: Dec. 4, 2003
(86) PCT No.: PCT/US03/38685

§ 371 Date: Apr. 24, 2006

Related U.S. Application Data
(60) Provisional application No. 60/430,982, filed on Dec. 4, 2002.

(51) Int. Cl. A61K 48/00; C12Q 1/68; C07H 21/02; C12P 21/06; C07F 9/6512

(52) U.S. Cl. 514/44; 514/81; 435/6; 435/69.1; 435/320.1; 435/325; 536/23.1; 544/243

(57) ABSTRACT

The invention relates to antisense oligonucleotides, compositions and methods useful for modulating the expression of TRPM2. The compositions comprise antisense oligonucleotides, particularly antisense oligonucleotides targeted to nucleic acids encoding TRPM2.

### TECHNICAL FIELD

This invention relates to antisense oligonucleotides targeted to specific nucleotide sequences. In particular, the invention pertains to antisense oligonucleotides targeted to the nucleic acid encoding the transient receptor potential (TRP) channel, TRPM2, and to their use for reducing cellular levels of TRPM2.

### BACKGROUND

TRPM2 is a member of the superfamily of transient receptor potential (TRP) channels. These channels are believed to have six transmembrane domains and intracellular amino- and carboxy-termini. According to a recent classification, TRP channels are grouped into three families based up on sequence homology and particular structural motifs (Harteneck et al., 2000, Trends Neurosci., 23:159; Montell et al., 2002, Mol. Cell., 9:229). TRPM2 belongs to family M, named after the founding member, melastatin. TRPM channels are characterized by complex structural sub-regions in their amino- and carboxy-termini, which carry additional functionality such as kinase activity (Ryazanov, 2002, FEBS Lett., 514:26).

There is limited information regarding the expression and function of TRPM2. High levels of expression were detected in the nervous system and lower levels in peripheral tissues such as bone marrow, spleen, lung and heart (Nagamine et al., 1998, Genomics, 54:124; Perraud et al., 2001, Nature, 411:595). TRPM2-mediated Ca²⁺ influx was activated by the second messenger, ADP-ribose, and other intracellular nucleotides in a heterologous expression system as well as in immunocytes (Perraud et al., 2001, Nature, 411:595; Sano et al., 2001, Science, 293:1327).

### SUMMARY

Antisense oligonucleotides can be targeted to specific nucleic acid molecules in order to reduce the expression of the target nucleic acid molecules. For example, antisense oligonucleotides directed at the TRPM2 mRNA could be used therapeutically to reduce the level of TRPM2 receptors in a patient suffering from chronic pain. An inherent challenge of generating antisense oligonucleotides, however, is identifying nucleic acid sequences that are useful targets for antisense molecules. Antisense oligonucleotides are often targeted to sequences within a target mRNA based on, for example, the function of the sequences (e.g., the translation start site, coding sequences, etc.). Such approaches often fail because in its native state, mRNA is generally not in a linear conformation. Typically, mRNAs are folded into complex secondary and tertiary structures, rendering sequences on the interior of such folded molecules inaccessible to antisense oligonucleotides. Only antisense molecules directed to accessible portions of an mRNA can effectively contact the mRNA and potentially bring about a desired result. TRPM2 antisense molecules that are useful to reduce levels of TRPM2 and alleviate pain therefore must be directed at accessible mRNA sequences. The invention described herein provides TRPM2 antisense oligonucleotides directed to accessible portions of a TRPM2 mRNA. These antisense oligonucleotides are therapeutically useful for reducing TRPM2 levels.

The invention features isolated antisense oligonucleotides consisting essentially of 10 to 50 nucleotides and compositions containing such antisense oligonucleotides. The oligonucleotide can specifically hybridize within an accessible region of the human TRPM2 mRNA in its native state, wherein the accessible region is defined by nucleotides 4276 through 4294, 3879 through 3896, 5661 through 5678, or 2821 through 2838 of SEQ ID NO:1. The antisense oligonucleotide of the invention also can inhibit the production of TRPM2.

In some embodiments, compositions include a plurality of isolated antisense oligonucleotides, wherein each antisense oligonucleotide specifically hybridizes within a different accessible region. In some embodiments, an antisense oligonucleotide of the invention includes a modified backbone, one or more non-natural internucleoside linkages, an oligonucleotide analog, one or more substituted sugar moieties, and/or nucleotide base modifications or nucleotide base substitutions.

The invention features isolated antisense oligonucleotides consisting essentially of 10 to 50 nucleotides and compositions containing such antisense oligonucleotides. The oligonucleotide can specifically hybridize within an accessible region of the rat TRPM2 mRNA in its native state, wherein the accessible region is defined by nucleotides 273 through 294, 1848 through 1878, 3759 through 3782, 481 through 501, 1971 through 1988, 2067 through 2084, 2165 through 2187, 4139 through 4161, or 4248 through 4270 of SEQ ID NO:2, and wherein the isolated antisense oligonucleotide inhibits the production of TRPM2.

The invention also features compositions containing such isolated antisense oligonucleotides. The composition can include a plurality of isolated antisense oligonucleotides, wherein each antisense oligonucleotide specifically hybridizes with a different accessible region.

In another aspect, the invention features an isolated oligonucleotide consisting essentially of the sequence of SEQ ID NO:3, SEQ ID NO:4, SEQ ID NO:5, SEQ ID NO:6, SEQ D NO:7, SEQ ID NO:8, SEQ ID NO:9, SEQ ID NO:10; SEQ ID NO:11; SEQ ID NO:12; SEQ ID NO:13; SEQ ID NO:14; or SEQ ID NO:15.

In yet another aspect, the invention features a method of decreasing production of TRPM2 in cells or tissues. The method includes contacting the cells or tissues with an antisense oligonucleotide that specifically hybridizes within an accessible region of TRPM2. The contacting step can result in an inhibition of pain sensory neurons.

The invention also features an nucleic acid construct that includes a regulatory element operably linked to a nucleic acid encoding a transcript, wherein the transcript specifically hybridizes within one or more accessible regions of TRPM2 mRNA in its native form, and host cells containing such nucleic acid constructs.

In yet another aspect, the invention features an isolated antisense oligonucleotide that specifically hybridizes within an accessible region of TRPM2 mRNA in its native form, and wherein the antisense oligonucleotide inhibits production of TRPM2.

In another aspect, the invention features a method for modulating pain in a mammal. Such a method includes administering an isolated antisense oligonucleotide of the invention to the mammal.

In another aspect, the invention features a method of identifying a compound that modulates pain in a mammal. Such a method includes contacting cells comprising a TRPM2 nucleic acid with a compound; and detecting the amount of TRPM2 RNA or TRPM2 polypeptide in or secreted from the cell. Generally, a difference in the amount of TRPM2 RNA or TRPM2 polypeptide produced in the presence of the compound compared to the amount of TRPM2 RNA or TRPM2 polypeptide produced in the absence of the compound is an indication that the compound modulates pain in the mammal. The amount of the TRPM2 RNA can be determined by Northern blotting, and the amount of the TRPM2 polypeptide can be determined by Western blotting. A representative compound is an antisense oligonucleotide that specifically hybridizes within an accessible region of TRPM2 mRNA in its native form and inhibits production of TRPM2.

In another aspect, the invention features a method of identifying a compound that modulates pain in a mammal. Such a method includes contacting cells comprising a TRPM2 nucleic acid with a compound; and detecting the activity of TRPM2 in or secreted from the cell. Generally, a difference in the activity of TRPM2 in the presence of the compound compared to the activity of TRPM2 in the absence of the compound is an indication that the compound modulates pain in the mammal.

In another aspect, the invention features a method for modulating pain in a mammal that includes administering a compound to the mammal that modulates the expression of TRPM2. A representative compound is an antisense oligonucleotide that specifically hybridizes within an accessible region of TRPM2 mRNA in its native form and inhibits expression of TRPM2. Typically, the pain is from diabetic neuropathy, gastric pain, postherpetic neuralgia, fibromyalgia, surgery, or chronic back pain.

In another aspect, the invention features a method for modulating pain in a mammal that includes administering a compound to the mammal that modulates the function of TRPM2. Typically, the pain is from diabetic neuropathy, gastric pain, postherpetic neuralgia, fibromyalgia, surgery, or chronic back pain.

In yet another aspect, the invention features methods for identifying a pain effector for TRPM2, the method including comparing the pain responsiveness of a test animal that contains TRPM2 that has been treated with a candidate effector with a control animal that does not contain TRPM2 that has been treated with a candidate effector.

In yet another aspect, the invention features methods for identifying a TRPM2 inhibitor, the method includes comparing the physiological response of a control cell that does not contain TRPM2 and that has been contacted with a candidate inhibitor with the physiological response of a test cell that contains TRPM2 and that has been contacted with a candidate inhibitor.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 shows the distribution of TRPM2 in human DRG and spinal cord.

FIG. 2 shows the distribution of TRPM2 in rat DRG and spinal cord.

FIG. 3 shows the pattern of TRPM2 expression before and after spinal nerve ligation.

FIG. 4 shows the effect of TRPM2 antisense oligonucleotides in a rat model of neuropathic pain.

FIG. 5 shows the nucleotide sequence of human TRPM2 (SEQ ID NO:1).

FIG. 6 shown the nucleotide sequence of rat TRPM2 (SEQ ID NO:2).

Like reference symbols in the various drawings indicate like elements.

### DETAILED DESCRIPTION

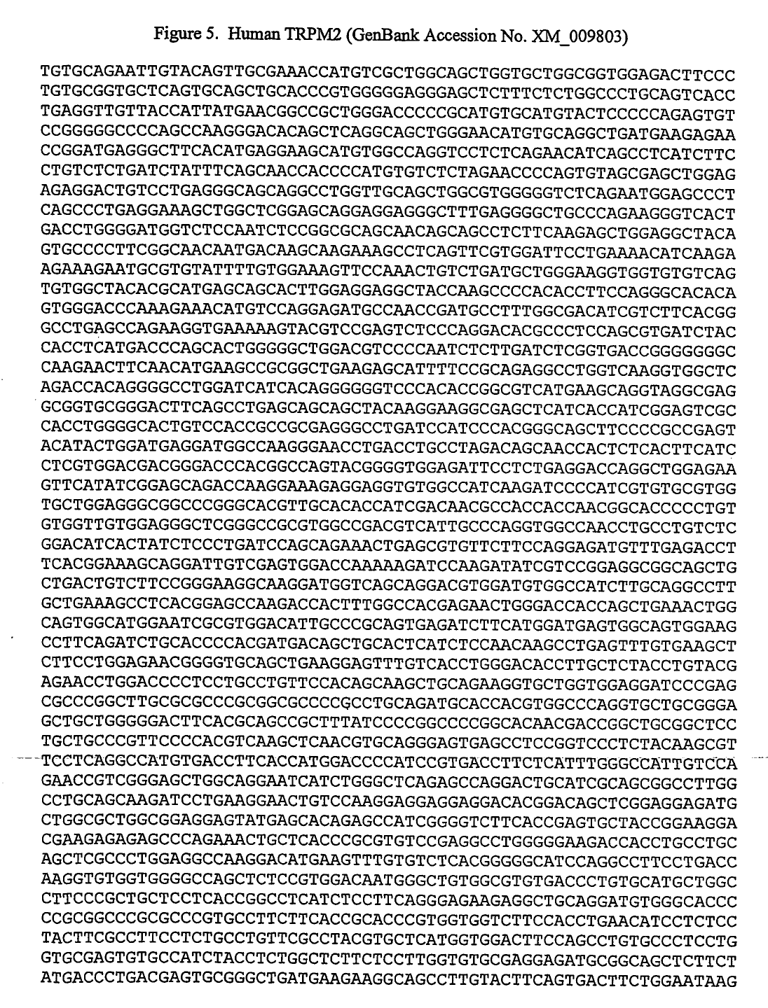

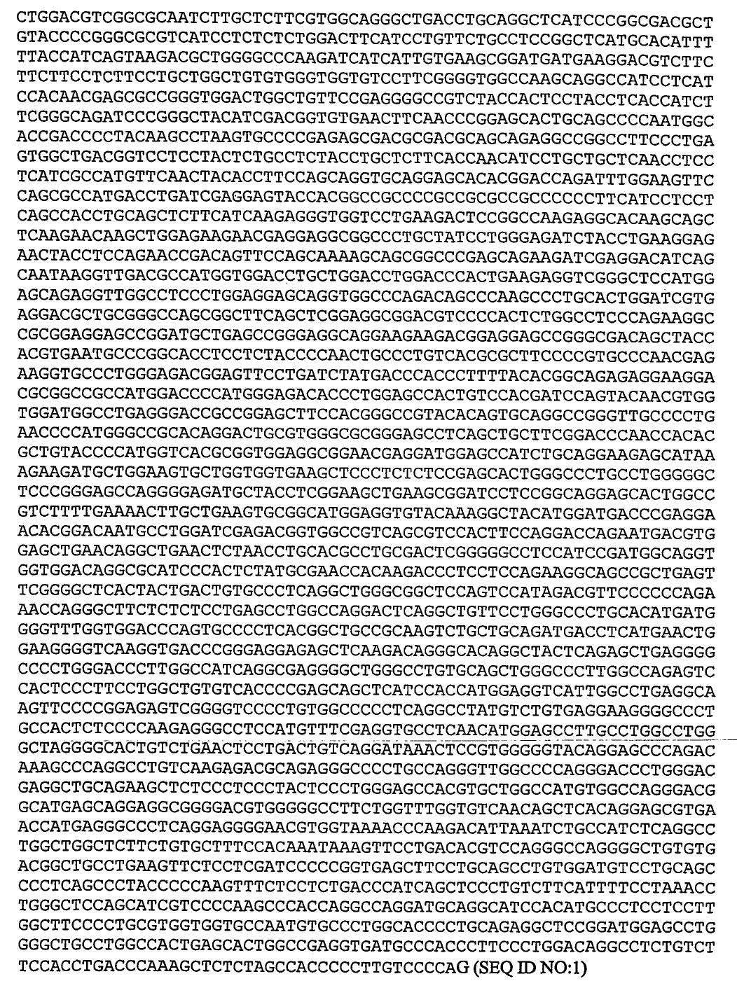

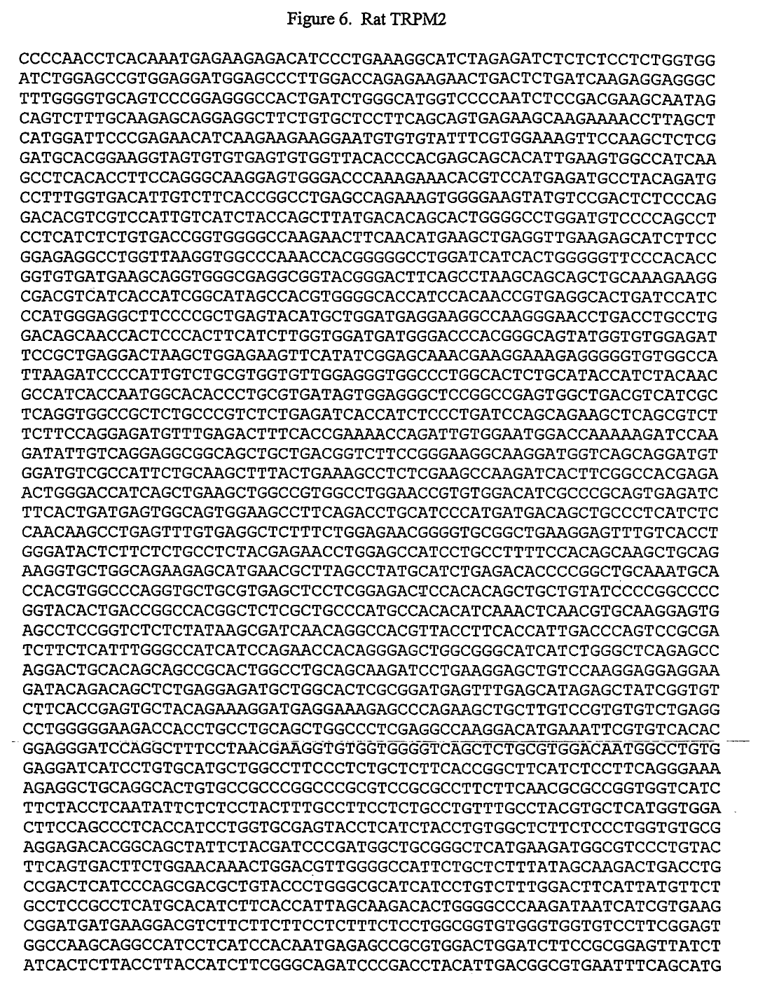

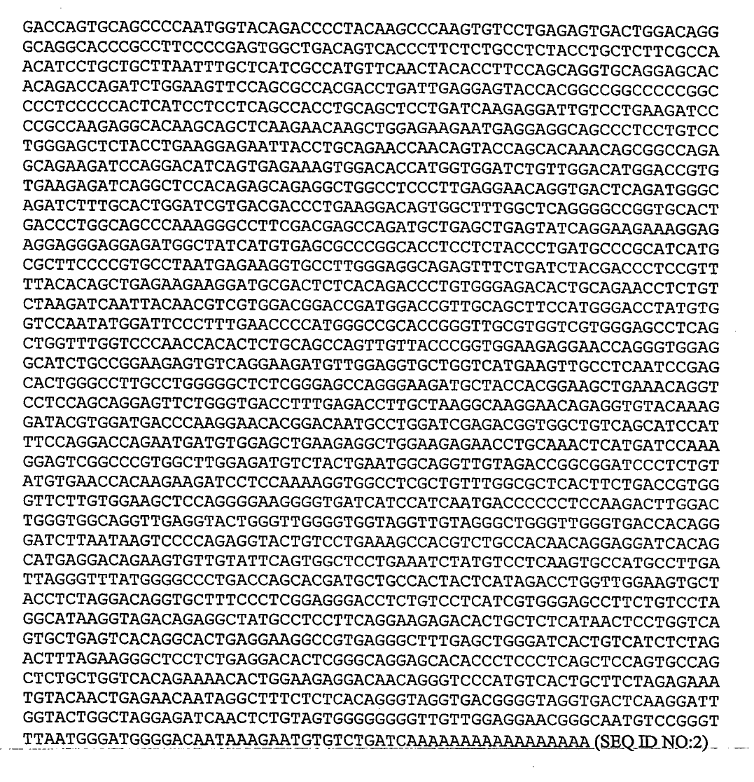

The present invention employs antisense compounds, particularly oligonucleotides, to modulate the function of target nucleic acid molecules. As used herein, the term “target nucleic acid” refers to both RNA and DNA, including cDNA, genomic DNA, and synthetic (e.g., chemically synthesized) DNA. The target nucleic acid can be double-stranded or single-stranded (i.e., a sense or an antisense single strand). In some embodiments, the target nucleic acid encodes a TRPM2 polypeptide. Thus, a “target nucleic acid” encompasses DNA encoding TRPM2, RNA (including pre-mRNA and mRNA) transcribed from such DNA, and also cDNA derived from such RNA. FIGS. 5 and 6 provide nucleic acid sequences that encode human and rat TRPM2 polypeptides, respectively (SEQ ID NO:1 and SEQ ID NO:2, respectively). An “antisense” compound is a compound containing nucleic acids or nucleic acid analogs that can specifically hybridize to a target nucleic acid, and the modulation of expression of a target nucleic acid by an antisense oligonucleotide is generally referred to as “antisense technology”.

The term “hybridization,” as used herein, means hydrogen bonding, which can be Watson-Crick, Hoogsteen, or reversed Hoogsteen hydrogen bonding, between complementary nucleoside or nucleotide bases. For example, adenine and thymine, and guanine and cytosine, respectively, are complementary nucleobases (often referred to in the art simply as “bases”) that pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide in a target nucleic acid molecule, then the oligonucleotide and the target nucleic acid are considered to be complementary to each other at that position. The oligonucleotide and the target nucleic acid are complementary to each other when a sufficient number of corresponding positions in each molecule are occupied by nucleotides that can hydrogen bond with each other. Thus, “specifically hybridizable” is used to indicate a sufficient degree of complementarity or precise pairing such that stable and specific binding occurs between the oligonucleotide and the target nucleic acid.

It is understood in the art that the sequence of an antisense oligonucleotide need not be 100% complementary to that of its target nucleic acid to be specifically hybridizable. An antisense oligonucleotide is specifically hybridizable when (a) binding of the oligonucleotide to the target nucleic acid interferes with the normal function of the target nucleic acid, and (b) there is sufficient complementarity to avoid non-specific binding of the antisense oligonucleotide to non-target sequences under conditions in which specific binding is desired, i.e., under conditions in which in vitro assays are performed or under physiological conditions for in vivo assays or therapeutic uses.

Stringency conditions in vitro are dependent on temperature, time, and salt concentration (see, e.g., Sambrook et al., Molecular Cloning: A Laboratory Manual, Cold Spring Harbor Laboratory Press, NY (1989)). Typically, conditions of high to moderate stringency are used for specific hybridization in vitro, such that hybridization occurs between substantially similar nucleic acids, but not between dissimilar nucleic acids. Specific hybridization conditions are hybridization in 5×SSC (0.75 M sodium chloride/0.075 M sodium citrate) for 1 hour at 40° C. with shaking, followed by washing 10 times in 1×SSC at 40° C. and 5 times in 1×SSC at room temperature. Oligonucleotides that specifically hybridize to a target nucleic acid can be identified by recovering the oligonucleotides from the oligonucleotide/target hybridization duplexes (e.g., by boiling) and sequencing the recovered oligonucleotides.

In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions, such as those used in the RiboTAG™ technology described below. For example, hybridization can be carried out in vitro in 2×SSC (0.3 M sodium chloride/0.03 M sodium citrate), 0.1% SDS at 37° C. A wash solution containing 4×SSC, 0.1% SDS can be used at 37° C., with a final wash in 1×SSC at 45° C.

The specific hybridization of an antisense molecule with its target nucleic acid can interfere with the normal function of the target nucleic acid. For a target DNA nucleic acid, antisense technology can disrupt replication and transcription. For a target RNA nucleic acid, antisense technology can disrupt, for example, translocation of the RNA to the site of protein translation, translation of protein from the RNA, splicing of the RNA to yield one or more mRNA species, and catalytic activity of the RNA. The overall effect of such interference with target nucleic acid function is, in the case of a nucleic acid encoding TRPM2, modulation of the expression of TRPM2. In the context of the present invention, “modulation” means a decrease in the expression of a gene (e.g., due to inhibition of transcription) and/or a decrease in cellular levels of the protein (e.g., due to inhibition of translation).

Identification of Target Sequences for TRPM2 Antisense Oligonucleotides

Antisense oligonucleotides are preferably directed at specific targets within a nucleic acid molecule. The process of “targeting” an antisense oligonucleotide to a particular nucleic acid usually begins with the identification of a nucleic acid sequence whose function is to be modulated. This nucleic acid sequence can be, for example, a gene (or mRNA transcribed from the gene) whose expression is associated with a particular disorder or disease state.

The targeting process also includes the identification of a site or sites within the target nucleic acid molecule where an antisense interaction can occur such that the desired effect, e.g., detection of TRPM2 mRNA or modulation of TRPM2 expression, will result. Traditionally, preferred target sites for antisense oligonucleotides have included the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions.

Simple knowledge of the sequence and domain structure (e.g., the location of translation initiation codons, exons, or introns) of a target nucleic acid, however, is generally not sufficient to ensure that an antisense oligonucleotide directed to a specific region will effectively bind to and modulate the function of the target nucleic acid. In its native state, an mRNA molecule is folded into complex secondary and tertiary structures, and sequences that are on the interior of such structures are inaccessible to antisense oligonucleotides. For maximal effectiveness, antisense oligonucleotides can be directed to regions of a target mRNA that are most accessible, i.e., regions at or near the surface of a folded mRNA molecule.

Accessible regions of an mRNA molecule can be identified by methods known in the art, including the use of RiboTAG™ technology. This technology is disclosed in PCT application number SE01/02054. In the RiboTAG™ method, also known as mRNA Accessible Site Tagging (MAST), oligonucleotides that can interact with a test mRNA in its native state (i.e., under physiological conditions) are selected and sequenced, thus leading to the identification of regions within the test mRNA that are accessible to antisense molecules. In a version of the RiboTAG™ protocol, the test mRNA is produced by in vitro transcription and is then immobilized, for example by covalent or non-covalent attachment to a bead or a surface (e.g., a magnetic bead). The immobilized test mRNA is then contacted by a population of oligonucleotides, wherein a portion of each oligonucleotide contains a different, random sequence. Oligonucleotides that can hybridize to the test mRNA under conditions of low stringency are separated from the remainder of the population (e.g., by precipitation of the magnetic beads). The selected oligonucleotides then can be amplified and sequenced; these steps of the protocol are facilitated if the random sequences within each oligonucleotide are flanked on one or both sides by known sequences that can serve as primer binding sites for PCR amplification.

In general, oligonucleotides that are useful in RiboTAG™ technology contain between 15 and 18 random bases, flanked on either side by known sequences. These oligonucleotides are contacted by the test mRNA under conditions that do not disrupt the native structure of the mRNA (e.g., in the presence of medium pH buffering, salts that modulate annealing, and detergents and/or carrier molecules that minimize non-specific interactions). Typically, hybridization is carried out at 37 to 40° C., in a solution containing 1× to 5×SSC and about 0.1% SDS. Non-specific interactions can be minimized further by blocking the known sequence(s) in each oligonucleotide with the primers that will be used for PCR amplification of the selected oligonucleotides.

As described herein, accessible regions of the nucleic acids encoding human and rat TRPM2 can be mapped. Particularly useful antisense oligonucleotides include those that specifically hybridize within accessible regions defined by nucleotides 4276 through 4294, 3879 through 3896, 5661 through 5678, or 2821 through 2838 of SEQ ID NO:1 or nucleotides 273 through 294, 1848 through 1878, 3759 through 3782, 481 through 501, 1971 through 1988, 2067 through 2084, 2165 through 2187, 4139 through 4161, or 4248 through 4270 of SEQ ID NO:2.

Non-limiting examples of such antisense oligonucleotides include the following nucleotide sequences:

| | |
|---|---|
| CGC GTC CTT CCT CTC TGC C; | (SEQ ID NO:3) |
| TGT CCT CGA TCT TCT GCT; | (SEQ ID NO:4) |
| ACG TCC CCG CCT CCT GCT; | (SEQ ID NO:5) |
| ACC ACC ACG GGT GCG GTG; | (SEQ ID NO:6) |
| CAT TCC TTC TTC TTG ATG TTC T; | (SEQ ID NO:7) |
| GAG TTT GAT GTG TGG CAT GGG CA; | (SEQ ID NO:8) |
| CTC CTC CCT CCT CTC CTT TCT TCC; | (SEQ ID NO:9) |
| TTC CCC ACT TTC TGG CTC AG; | (SEQ ID NO:10) |
| GCT CCC TGT GGT TCT GGA; | (SEQ ID NO:11) |
| TAT CTT CCT CCT CCT TGG; | (SEQ ID NO:12) |
| TTC TGG GCT CTT TCC TCA TCC TT; | (SEQ ID NO:13) |
| CCT CCA CCC TGG TTC CTC TTC CA; | (SEQ ID NO:14) |
| and | |
| TAG CAT CTT CCC TGG CTC CCG AG. | (SEQ ID NO:15) |

It should be noted that an antisense oligonucleotide may consist essentially of a nucleotide sequence that specifically hybridizes with an accessible region set out above. Such antisense oligonucleotides, however, may contain additional flanking sequences of 5 to 10 nucleotides at either end. Flanking sequences can include, for example, additional sequence of the target nucleic acid or primer sequence.

For maximal effectiveness, further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs).

TRPM2 Antisense Oligonucleotides

Once one or more target sites have been identified, antisense oligonucleotides can be synthesized that are sufficiently complementary to the target (i.e., that hybridize with sufficient strength and specificity to give the desired effect). In the context of the present invention, the desired effect is the modulation of TRPM2 expression such that cellular TRPM2 levels are reduced. The effectiveness of an antisense oligonucleotide to modulate expression of a target nucleic acid can be evaluated by measuring levels of the mRNA or protein products of the target nucleic acid (e.g., by Northern blotting, RT-PCR, Western blotting, ELISA, or immunohistochemical staining).

In some embodiments, it may be useful to target multiple accessible regions of a target nucleic acid. In such embodiments, multiple antisense oligonucleotides can be used that each specifically hybridize to a different accessible region. Multiple antisense oligonucleotides can be used together or sequentially.

The antisense oligonucleotides in accordance with this invention can be from about 10 to about 50 nucleotides in length (e.g., 12 to 40, 14 to 30, or 15 to 25 nucleotides in length). Antisense oligonucleotides that are 15 to 23 nucleotides in length are particularly useful. However, an antisense oligonucleotide containing even fewer than 10 nucleotides (for example, a portion of one of the preferred antisense oligonucleotides) is understood to be included within the present invention so long as it demonstrates the desired activity of inhibiting expression of the P2X₃ purinoreceptor.

An “antisense oligonucleotide” can be an oligonucleotide as described herein. The term “oligonucleotide” refers to an oligomer or polymer of ribonucleic acid (RNA) or deoxyribonucleic acid (DNA) or analogs thereof. This term includes oligonucleotides composed of naturally occurring nucleobases, sugars and covalent intemucleoside (backbone) linkages, as well as oligonucleotides having non-naturally occurring portions which function similarly. Such modified or substituted oligonucleotides are often preferred over native forms because of desirable properties such as, for example, enhanced cellular uptake, enhanced affinity for a nucleic acid target, and increased stability in the presence of nucleases.

While antisense oligonucleotides are a preferred form of antisense compounds, the present invention includes other oligomeric antisense compounds, including but not limited to, oligonucleotide analogs such as those described below. As is known in the art, a nucleoside is a base-sugar combination, wherein the base portion is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be further joined to form a circular structure, although linear structures are generally preferred. Within the oligonucleotide structure, the phosphate groups are commonly referred to as forming the intemucleoside backbone of the oligonucleotide. The normal linkage or backbone of RNA and DNA is a 3′ to 5′ phosphodiester linkage.

TRPM2 antisense oligonucleotides that are useful in the present invention include oligonucleotides containing modified backbones or non-natural intemucleoside linkages. As defined herein, oligonucleotides having modified backbones include those that have a phosphorus atom in the backbone and those that do not have a phosphorus atom in the backbone. For the purposes of this specification, and as sometimes referenced in the art, modified oligonucleotides that do not have a phosphorus atom in their intemucleoside backbone also can be considered to be oligonucleotides.

Modified oligonucleotide backbones can include, for example, phosphorothioates, chiral phosphorothioates, phosphorodithioates, phosphotriesters, aminoalkylphosphotriesters, methyl and other alkyl phosphonates (e.g., 3′-alkylene phosphonates and chiral phosphonates), phosphinates, phosphoramidates (e.g., 3′-amino phosphoramidate and aminoalkylphosphoramidates), thionophosphoramidates, thionoalkylphosphonates, thionoalkylphosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. References that teach the preparation of such modified backbone oligonucleotides are provided, for example, in U.S. Pat. Nos. 4,469,863 and 5,750,666.

TRPM2 antisense molecules with modified oligonucleotide backbones that do not include a phosphorus atom therein can have backbones that are formed by short chain alkyl or cycloalkyl intemucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. References that teach the preparation of such modified backbone oligonucleotides are provided, for example, in U.S. Pat. Nos. 5,235,033 and 5,596,086.

In another embodiment, a TRPM2 antisense compound can be an oligonucleotide analog, in which both the sugar and the internucleoside linkage (i.e., the backbone) of the nucleotide units are replaced with novel groups, while the base units are maintained for hybridization with an appropriate nucleic acid target. One such oligonucleotide analog that has been shown to have excellent hybridization properties is referred to as a peptide nucleic acid (PNA). In PNA compounds, the sugar-backbone of an oligonucleotide is replaced with an amide containing backbone (e.g., an aminoethylglycine backbone). The nucleobases are retained and are bound directly or indirectly to aza nitrogen atoms of the amide portion of the backbone. References that teach the preparation of such modified backbone oligonucleotides are provided, for example, in Nielsen et al., 1991, Science, 254:1497-1500, and in U.S. Pat. No. 5,539,082.

Other useful TRPM2 antisense oligonucleotides can have phosphorothioate backbones and oligonucleosides with heteroatom backbones, and in particular CH₂NHOCH₂, CH₂N(CH₃)OCH₂, CH₂ON(CH₃)CH₂, CH₂N(CH₃)N(CH₃)CH₂, and ON(CH₃)CH₂CH₂ (wherein the native phosphodiester backbone is represented as OPOCH₂) as taught in U.S. Pat. No. 5,489,677, and the amide backbones disclosed in U.S. Pat. No. 5,602,240.

Substituted sugar moieties also can be included in modified oligonucleotides. TRPM2 antisense oligonucleotides of the invention can comprise one or more of the following at the 2′ position: OH; F; O—, S—, or N-alkyl; O—, S—, or N-alkenyl; O—, S—, or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Useful modifications also can include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(C₂)ₙCH₃)]₂, where n and m are from 1 to about 10. In addition, oligonucleotides can comprise one of the following at the 2′ position: C₁ to C₁₀ lower alkyl, substituted lower alkyl, alkaryl, aralkyl, O-alkaryl or O-aralkyl SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, groups for improving the pharmacokinetic or pharmacodynamic properties of an oligonucleotide, and other substituents having similar properties. Other useful modifications include an alkoxyalkoxy group, e.g., 2′-methoxyethoxy (2′-OCH₂CH₂OCH₃), a dimethylaminooxyethoxy group (2′-O(CH₂)₂ON(CH₃)₂), or a dimethylamino-ethoxyethoxy group (2′-OCH₂OCH₂N(CH₂)₂). Other modifications can include 2′-methoxy (2′-OCH₃), 2′-aminopropoxy (2′-OCH₂CH₂CH₂NH₂), or 2′-fluoro (2′-F). Similar modifications also can be made at other positions within the oligonucleotide, such as the 3′ position of the sugar on the 3′ terminal nucleotide or in 2′-5′ linked oligonucleotides, and the 5′ position of the 5′ terminal nucleotide. Oligonucleotides also can have sugar mimetics such as cyclobutyl moieties in place of the pentofuranosyl group. References that teach the preparation of such substituted sugar moieties include U.S. Pat. Nos. 4,981,957 and 5,359,044.

Useful TRPM2 antisense oligonucleotides also can include nucleobase modifications or substitutions. As used herein, “unmodified” or “natural” nucleobases include the purine bases adenine (A) and guanine (G), and the pyrimidine bases thymine (I), cytosine (C), and uracil (U). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Certain nucleobase substitutions can be particularly useful for increasing the binding affinity of the antisense oligonucleotides of the invention. For example, 5-methylcytosine substitutions have been shown to increase nucleic acid duplex stability by 0.6 to 1.2° C. (Sanghvi et al., eds., 1993, Antisense Research and Applications, pp. 276-278, CRC Press, Boca Raton, Fla.). Other useful nucleobase substitutions include 5-substituted pyrimidines, 6-azapyrimidines and N-2, N-6 and O-6 substituted purines such as 2-aminopropyladenine, 5-propynyluracil and 5-propynylcytosine.

Antisense oligonucleotides of the invention also can be modified by chemical linkage to one or more moieties or conjugates that enhance the activity, cellular distribution or cellular uptake of the oligonucleotide. Such moieties include but are not limited to lipid moieties (e.g., a cholesterol moiety); cholic acid; a thioether moiety (e.g., hexyl-S-tritylthiol); a thiocholesterol moiety, an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol chain; adamantane acetic acid; a palmityl moiety, or an octadecylamine or hexylamino-carbonyl-oxycholesterol moiety. The preparation of such oligonucleotide conjugates is disclosed in, for example, U.S. Pat. Nos. 5,218,105 and 5,214,136.

It is not necessary for all nucleobase positions in a given antisense oligonucleotide to be uniformly modified. More than one of the aforementioned modifications can be incorporated into a single oligonucleotide or even at a single nucleoside within an oligonucleotide. The present invention also includes antisense oligonucleotides that are chimeric oligonucleotides. “Chimeric” antisense oligonucleotides can contain two or more chemically distinct regions, each made up of at least one monomer unit (e.g., a nucleotide in the case of an oligonucleotide). Chimeric oligonucleotides typically contain at least one region wherein the oligonucleotide is modified so as to confer, for example, increased resistance to nuclease degradation, increased cellular uptake, and/or increased affinity for the target nucleic acid. For example, a region of a chimeric oligonucleotide can serve as a substrate for an enzyme such as RNase H, which is capable of cleaving the RNA strand of an RNA:DNA duplex such as that formed between a target mRNA and an antisense oligonucleotide. Cleavage of such a duplex by RNase H, therefore, can greatly enhance the effectiveness of an antisense oligonucleotide.

Antisense molecules in accordance with the invention can include enzymatic ribonucleic acid molecules that can cleave other ribonucleic acid molecules (ribozymes). Antisense technologies involving ribozymes have shown great utility in research, diagnostic and therapeutic contexts. Methods for designing and using ribozymes are well known, and have been extensively described. Ribozymes in general are described, for example, in U.S. Pat. Nos. 5,254,678; 5,496,698; 5,525,468; and 5,616,459. U.S. Pat. Nos. 5,874,414 and 6,015,794 describe trans-splicing ribozymes. Hairpin ribozymes are described, for example, in U.S. Pat. Nos. 5,631,115; 5,631,359; 5,646,020; 5,837,855 and 6,022,962. U.S. Pat. No. 6,307,041 describes circular, hairpin, circular/hairpin, lariat, and hairpin-lariat hammerhead ribozymes. Ribozymes can include deoxyribonucleotides (see e.g., U.S. Pat. Nos. 5,652,094; 6,096,715 and 6,140,491). Such ribozymes are often referred to as (nucleozymes). Ribozymes can include modified ribonucleotides. Base-modified enzymatic nucleic acids are described, for example, in U.S. Pat. Nos. 5,672,511; 5,767,263; 5,879,938 and 5,891,684. U.S. Pat. No. 6,204,027 describes ribozymes having 2′-O substituted nucleotides in the flanking sequences. U.S. Pat. No. 5,545,729 describes stabilized ribozyme analogs. Other ribozymes having specialized properties have been described, for example, in U.S. Pat. No. 5,942,395 (describing chimeric ribozymes that include a snoRNA stabilizing motif), U.S. Pat. Nos. 6,265,167 and 5,908,779 (describing nuclear ribozymes), U.S. Pat. No. 5,994,124 (describing ribozyme-snRNA chimeric molecules having a catalytic activity for nuclear RNAs); and U.S. Pat. No. 5,650,502 (describing ribozyme analogs with rigid non-nucleotidic linkers).

The TRPM2 antisense oligonucleotides of the invention are synthesized in vitro and do not include antisense compositions of biological origin, except for oligonucleotides that comprise the subject antisense oligonucleotides and that have been purified from or isolated from biological material. Antisense oligonucleotides used in accordance with this invention can be conveniently produced through the well-known technique of solid phase synthesis. Equipment for such synthesis is commercially available from several vendors including, for example, Applied Biosystems (Foster City, Calif.). Any other means for such synthesis known in the art additionally or alternatively can be employed. Similar techniques also can be used to prepare modified oligonucleotides such as phosphorothioates or alkylated derivatives.

Antisense Preparations and Methods for Use

The antisense oligonucleotides of the invention are useful for research and diagnostics, and for therapeutic use. For example, assays based on hybridization of antisense oligonucleotides to nucleic acids encoding TRPM2 can be used to evaluate levels of TRPM2 in a tissue sample. Hybridization of the antisense oligonucleotides of the invention with a nucleic acid encoding TRPM2 can be detected by means known in the art. Such means can include conjugation of an enzyme to the antisense oligonucleotide, radiolabeling of the antisense oligonucleotide, or any other suitable means of detection.

Antisense molecules in accordance with the invention also can be used in screening assays to identify small molecule therapeutics (effectors) that could be useful for the prophylactic or therapeutic treatment of pain. Such effectors could bind to, inhibit or stimulate TRPM2. Candidate effectors can be pre-existing natural compounds as well as known or new synthetic compounds. Candidate effectors can exist in collections with other compounds (e.g., in a chemical or peptide library). Candidate effectors can be provided using, for example, combinatorial library approaches known in the art, including: biological libraries; spatially addressable parallel solid phase or solution phase libraries; synthetic library methods requiring deconvolution; the “one-bead one-compound” library method; and synthetic library methods using affinity chromatography selection. Biological library approaches typically provide peptide libraries, while other approaches can provide peptide, non-peptide oligomet or small molecule libraries of compounds (see e.g., Lam, 1997, Anticancer Drug Des., 12:145). Methods for synthesizing such molecular libraries are well known and have been extensively described. See e.g., DeWitt et al., 1993, Proc. Natl. Acad Sci USA, 90:6909; Erb et al., 1994, Proc. Natl. Acad Sci. USA, 91:11422; Zuckermann et al., 1994, J. Med Chem., 37:2678; Cho et al., 1993, Science, 261:1303; Carrell et al., 1994, Angew. Chem. Int. Ed. Engl., 33:2059; Carell et al., 1994, Angew. Chem. Int. Ed Engl., 33:2061; and in Gallop et al., 1994, J. Med Chem., 37:1233. Molecular libraries can be provided in solution (see e.g., Houghten, 1992, Biotechniques, 13:412-21), on beads (see e.g., Lam, 1991, Nature, 354:82-84), on chips (see e.g., Fodor, 1993, Nature, 364:555-556), using bacteria (see e.g, U.S. Pat. No. 5,223,409), using spores (see e.g, U.S. Pat. No. 5,223,409), using plasmids (see e.g., Cull et al., 1992, Proc Natl Acad Sci USA, 89:1865-1869) and using phage (see e.g., U.S. Pat. No. 5,223,409; Scott and Smith, 1990, Science, 249:386-390; Devlin, 1990, Science, 249:404406; Cwirla et al., 1990, Proc. Natl. Acad. Sci. USA, 87:6378-6382; and Felici, 1991, J. Mol. Biol., 222:301-310).

Screening assays generally involve administering a candidate effector to a test animal or cell line that contains TRPM2. An effect observed in the test animal or cell line, if any, can be compared to that observed in a control animal or cell line that does not contain TRPM2. A control animal or cell line can be a null or “knockout” mutant in which the gene that encodes TRPM2 has been mutated such that it is not expressed. Animals or cell lines that contain one or more antisense molecules that specifically hybridize to an accessible region of TRPM2 mRNA also can be used as control animals. Animals or cell lines that contain a nucleic acid construct having a regulatory element operably linked to a nucleic acid that encodes a transcript that specifically hybridizes to an accessible region of TRPM2 mRNA also can be used as control animals.

Effects that can be determined in test and control animals include effects related to pain (e.g., responsiveness to pain sensation). Effects that can be determined in test and control cell lines include effects related to the level of TRPM2 mRNA or protein.

Effectors identified by the above-described screening assays can be used in an appropriate animal model to, for example, determine their efficacy, toxicity, or side effects. Effectors identified by the above-described screening assays also can be used in an animal model to determine their mechanism of action. Appropriate effectors can be used to treat health conditions that can be improved by modulating the activity of TRPM2. Such health conditions could be associated with abnormal expression or activity of TRPM2.

Those of skill in the art also can harness the specificity and sensitivity of antisense technology for therapeutic use. Antisense oligonucleotides have been employed as therapeutic moieties in the treatment of disease states in animals, including humans. For therapeutic methods, the cells or tissues are typically within a vertebrate (e.g., a mammal such as a human.

The invention provides therapeutic methods for treating conditions arising from abnormal expression (e.g., over-production) of the TRPM2 purinoreceptor. By these methods, antisense oligonucleotides in accordance with the invention are administered to a subject (e.g., a human) suspected of having a disease or disorder (e.g., chronic pain) that can be alleviated by modulating the expression of TRPM2. Typically, one or more antisense oligonucleotides can be administered to a subject suspected of having a disease or condition associated with the expression of TRPM2. The antisense oligonucleotide can be in a pharmaceutically acceptable carrier or diluent, and can be administered in amounts and for periods of time that will vary depending upon the nature of the particular disease, its severity, and the subject's overall condition. Typically, the antisense oligonucleotide is administered in an inhibitory amount (i.e., in an amount that is effective for inhibiting the production of TRPM2 in the cells or tissues contacted by the antisense oligonucleotides). The antisense oligonucleotides and methods of the invention also can be used prophylactically, e.g., to minimize pain in a subject known to have high levels of TRPM2.

The ability of a TRPM2 antisense oligonucleotide to inhibit expression and/or production of TRPM2 can be assessed, for example, by measuring levels of TRPM2 mRNA or protein in a subject before and after treatment. Methods for measuring mRNA and protein levels in tissues or biological samples are well known in the art. If the subject is a research animal, for example, TRPM2 levels in the brain can be assessed by in situ hybridization or immunostaining following euthanasia. Indirect methods can be used to evaluate the effectiveness of TRPM2 antisense oligonucleotides in live subjects. For example, reduced expression of TRPM2 can be inferred from reduced sensitivity to painful stimuli. As described in the Examples below, animal models can be used to study the development, maintenance, and relief of chronic neuropathic or inflammatory pain. Animals subjected to these models generally develop thermal hyperalgesia (i.e., an increased response to a stimulus that is normally painful) and/or allodynia (i.e., pain due to a stimulus that is not normally painful). Sensitivity to mechanical and thermal stimuli can be assessed (see Bennett, 2001, Methods in Pain Research, pp. 67-91, Kruger, ed.) to evaluate the effectiveness of TRPM2 antisense treatment.

Methods for formulating and subsequently administering therapeutic compositions are well known to those skilled in the art. See, for example, Remington, 2000, The Science and Practice of Pharmacy, 20^{th} Ed., Gennaro & Gennaro, eds., Lippincott, Williams & Wilkins. Dosing is generally dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual oligonucleotides, and can generally be estimated based on EC₅₀ found to be effective in in vitro and in vivo animal models. Typically, dosage is from 0.01 μg to 100 g per kg of body weight, and may be given once or more daily, weekly, or even less often. Following successful treatment, it may be desirable to have the patient undergo maintenance therapy to prevent the recurrence of the disease state.

The present invention provides pharmaceutical compositions and formulations that include the TRPM2 antisense oligonucleotides of the invention. TRPM2 antisense oligonucleotides therefore can be admixed, encapsulated, conjugated or otherwise associated with other molecules, molecular structures, or mixtures of oligonucleotides such as, for example, liposomes, receptor targeted molecules, or oral, rectal, topical or other formulations, for assisting in uptake, distribution and/or absorption.

A “pharmaceutically acceptable carrier” (also referred to herein as an “excipient”) is a pharmaceutically acceptable solvent, suspending agent, or any other pharmacologically inert vehicle for delivering one or more therapeutic compounds (e.g., TRPM2 antisense oligonucleotides) to a subject. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with nucleic acids include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

The pharmaceutical compositions of the present invention can be administered by a number of methods depending upon whether local or systemic treatment is desired and upon the area to be treated. Administration can be, for example, topical (e.g., transdermal, ophthalmic, or intranasal); pulnonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, antisense oligonucleotides can be administered by injection or infusion into the cerebrospinal fluid, preferably with one or more agents capable of promoting penetration of the antisense oligonucleotide across the blood-brain barrier.

Formulations for topical administration of antisense oligonucleotides include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Coated condoms, gloves and the like also may be useful. Conventional pharmaceutical carriers, aqueous, powder or oily bases, thickeners and the like may be necessary or desirable.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Oligonucleotides with at least one 2′-O-methoxyethyl modification (described above) may be particularly useful for oral administration.

Compositions and formulations for parenteral, intrathecal or intraventricular administration can include sterile aqueous solutions, which also can contain buffers, diluents and other suitable additives (e.g., penetration enhancers, carrier compounds and other pharmaceutically acceptable carriers).

Pharmaceutical compositions of the present invention include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain, the antisense composition to be delivered. Liposomes can be particularly useful from the standpoint of drug delivery due to their specificity and the duration of action they offer. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

The TRPM2 antisense oligonucleotides of the invention further encompass any pharmaceutically acceptable salts, esters, or salts of such esters, or any other compound which, upon administration to an animal including a human, is capable of providing (directly or indirectly) the biologically active metabolite or residue thereof. Accordingly, for example, the invention provides pharmaceutically acceptable salts of TRPM2 antisense oligonucleotides, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the oligonucleotides of the invention (i.e., salts that retain the desired biological activity of the parent oligonucleotide without imparting undesired toxicological effects). Examples of pharmaceutically acceptable salts of oligonucleotides include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed from elemental anions (e.g., chlorine, bromine, and iodine).

Pharmaceutical compositions containing the antisense oligonucleotides of the present invention also can incorporate penetration enhancers that promote the efficient delivery of nucleic acids, particularly oligonucleotides, to the skin of animals. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas).

Certain embodiments of the invention provide pharmaceutical compositions containing (a) one or more antisense oligonucleotides and (b) one or more other agents that function by a non-antisense mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions of the invention. Other non-antisense agents (e.g., chemotherapeutic agents) are also within the scope of this invention. Such combined compounds can be used together or sequentially.

The antisense compositions of the present invention additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions of the present invention, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the antisense components within the compositions of the present invention. The formulations can be sterilized and, if desired, and the like which do not deleteriously interact with the nucleic acid(s) of the formulation.

The pharmaceutical formulations of the present invention, which can be presented conveniently in unit dosage form, can be prepared according to conventional techniques well known in the pharmaceutical industry. Such techniques include the step of bringing into association the active ingredients (e.g., the TRPM2 antisense oligonucleotides of the invention) with the desired pharmaceutical carrier(s) or excipient(s). Typically, the formulations can be prepared by uniformly bringing the active ingredients into intimate association with liquid carriers or finely divided solid carriers or both, and then, if necessary, shaping the product. Formulations can be sterilized if desired, provided that the method of sterilization does not interfere with the effectiveness of the antisense oligonucleotide contained in the formulation.

The compositions of the present invention can be formulated into any of many possible dosage forms such as, but not limited to, tablets, capsules, liquid syrups, soft gels, suppositories, and enemas. The compositions of the present invention also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

Nucleic Acid Constructs

Nucleic acid constructs (e.g., a plasmid vector) are capable of transporting a nucleic acid into a host cell. Suitable host cells include prokaryotic or eukaryotic cells (e.g., bacterial cells such as E. coli, insect cells, yeast cells, and mammalian cells). Some constructs are capable of autonomously replicating in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and are replicated with the host genome.

Nucleic acid constructs can be, for example, plasmid vectors or viral vectors (e.g., replication defective retroviruses, adenoviruses, and adeno-associated viruses). Nucleic acid constructs include one or more regulatory sequences operably linked to the nucleic acid of interest (e.g., a nucleic acid encoding a transcript that specifically hybridizes to a TRPM2 mRNA in its native form). With respect to regulatory elements, “operably linked” means that the regulatory sequence and the nucleic acid of interest are positioned such that nucleotide sequence is transcribed (e.g., when the vector is introduced into the host cell).

Regulatory sequences include promoters, enhancers and other expression control elements (e.g., polyadenylation signals). (See, e.g., Goeddel, 1990, Gene Expression Technology: Methods in Enzymology, 185, Academic Press, San Diego, Calif.). Regulatory sequences include those that direct constitutive expression of a nucleotide sequence in many types of host cells and that direct expression of the nucleotide sequence only in certain host cells (e.g., cell type or tissue-specific regulatory sequences).

Transgenic organisms and stable cell lines comprising antisense molecules and nucleic acid constructs according to the invention can be made as a matter of routine by those of skill in the art. A number-of references in the literature describe the expression of heterologous genes in cells of bacteria, yeast, filamentous fungi, plants, insects, and mammals, including humans and other primates, rodents, rabbits, swine, and bovines. Numerous references that describe the production of such transgenic organisms and stable cell lines include, but are not limited to U.S. Pat. Nos. 6,156,192; 4,736,866; and 6,271,436. Transgenic organisms and cell lines in accordance with the invention also can be obtained from numerous commercial sources on a fee-for-service basis.

Articles of Manufacture

Antisense oligonucleotides of the invention can be combined with packaging material and sold as kits of reducing TRPM2 expression. Components and methods for producing articles of manufacture are well known. The articles of manufacture may combine one or more of the antisense oligonucleotides set out in the above sections. In addition, the article of manufacture further may include buffers, hybridization reagents, or other control reagents for reducing or monitoring reduced TRPM2 expression. Instructions describing how the antisense oligonucleotides are effective for reducing TRPM2 expression can be included in such kits.

The invention will be further described in the following examples, which do not limit the scope of the invention described in the claims.

### EXAMPLES

### Example 1

### Spinal Catheterization

Male Sprague Dawley (Harlan, Ind.) rats weighing between 200 and 250 g obtained from Harlan (Indianapolis, Ind.) were deeply anesthetized with a mixture containing 75 mg/kg ketamine, 5 mg/kg xylazine, and 1 mg/kg acepromazine, and a catheter (8.5 cm; PE-10) was passed to the lumbosacral intrathecal space through an incision in the dura over the atlantooccipital joint. Following surgery, animals were kept on a warming blanket and were periodically turned and carefully observed until completely recovered from anesthesia. Animals were allowed to recover for 3 days before being subjected to models of chronic pain.

### Example 2

### Mechanical Nociceptive Testing

Baseline, post-injury, and post-treatment values for mechanical sensitivity were evaluated with calibrated monofilaments (von Frey filaments) according to the up-down method (Chaplan et al., 1994, J. Neurosci. Methods, 53:55-63). Animals were placed on a wire mesh platform and allowed to acclimate to their surroundings for a minimum of 15 minutes before testing. Filaments of increasing force were sequentially applied to the plantar surface of the paw just to the point of bending, and held for three seconds. The behavioral endpoint of the stimulus (achieved when the stimulus was of sufficient force) was the point at which the animal would lick, withdraw and/or shake the paw. The force or pressure required to cause a paw withdrawal was recorded as a measure of threshold to noxious mechanical stimuli for each hind-paw. The mean and standard error of the mean (SEM) were determined for each animal in each treatment group. The data were analyzed using repeated measures ANOVA followed by the Bonferonni post-hoc test. Since this stimulus is normally not considered painful, significant injury-induced increases in responsiveness in this test were interpreted as a measure of mechanical allodynia.

### Example 3

### Thermal Nociceptive Testing

Baseline, post-injury, and post-treatment thermal sensitivities are determined by measuring withdrawal latencies in response to radiant heat stimuli delivered to the plantar surface of the hind-paws (Hargreaves et al., 1988, Pain, 32:77-88). Animals are placed on a plexiglass platform and allowed to acclimate for a minimum of 10 minutes. A radiant heat source is directed to the plantar surface, and the time to withdrawal is measured. For each paw, the withdrawal latency is determined by averaging three measurements separated by at least 5 minutes. The heating device is set to automatically shut off after a programmed period of time to avoid damage to the skin of unresponsive animals. The data is analyzed using repeated measures ANOVA followed by the Bonferonni post-hoc test. Significant injury-induced increases in thermal response latencies are considered to be a measure of thermal hyperalgesia since the stimulus is normally in the noxious range.

### Example 4

### Induction of Chronic-Neuropathic Pain

The Spinal Nerve Ligation (SNL) model (Kim & Chung, 1992, Pain, 50:355-63) was used to induce chronic neuropathic pain. Rats were anesthetized with isoflurane, the left L5 transverse process was removed, and the L5 and L6 spinal nerves were tightly ligated with 6-0 silk suture. The wound was then closed with internal sutures and external staples. Control animals received a sham surgery consisting of removing the transverse process and exposing the L5 spinal nerve without ligating. All operations were performed on the left side of the animal.

### Example 5

### Induction of Chronic Inflammation

The complete Freund's adjuvant (CFA) model of chronic peripheral inflammation is utilized (see, for example, Hylden et al., 1989, Pain, 37:229-43). Rats under light anesthesia receive an injection of CFA (75 μl) into the left hindpaw using a sterile 1.0 ml syringe. A separate population of control rats is subjected to unilateral injection of saline.

### Example 6

### Antisense Design and Injection

Oligonucleotides were commercially synthesized (Midland Certified Reagent Company, Midland, Tex.) and dissolved in dH₂O. Oligonucleotides were delivered into the intrathecal space either in a volume of 5 μl per injection twice daily for 3 to 4 days as previously described (see, for example, Bilsky et al., 1996, Neurosci. Lett., 220:155-158; Bilsky et al., 1996, J. Pharmacol. Exp. Ther., 277:491-501; and Vanderah et al., 1994, Neuroreport., 5:2601-2605) or by slow infusion by osmotic minipump. The antisense oligonucleotides shown in Tables 1 and 2 were designed. Random oligonucleotides were used as controls.

TABLE 1
RiboTAG-Designed Human Antisense Oligonucleotide Sequences

| | |
|---|---|
| Location on mRNA | SEQ ID |

| | | | | |
|---|---|---|---|---|
| Name | Antisense 3′ | Antisense 5′ | Antisense Sequence | NO: |

| | | | | |
|---|---|---|---|---|
| hTRPM2-1 | 4294 | 4276 | CGC GTC CTT CCT CTC TGC C | 3 |
| hTRPM2-2 | 3896 | 3879 | TGT CCT CGA TCT TCT GCT | 4 |
| hTRPM2-3 | 5678 | 5661 | ACG TCC CCG CCT CCT GCT | 5 |
| hTRPM2-4 | 2838 | 2821 | ACC ACC ACG GGT GCG GTG | 6 |

TABLE 2
RiboTAG-Designed Rat Antisense Oligonucleotide Sequences

| | |
|---|---|
| Location on mRNA | SEQ ID |

| | | | | |
|---|---|---|---|---|
| Name | Antisense 3′ | Antisense 5′ | Antisense Sequence | NO: |

| | | | | |
|---|---|---|---|---|
| rTRPM2-1 | 294 | 273 | CAT TCC TTC TTC TTG ATG TTC T | 7 |
| rTRPM2-2 | 1878 | 1848 | GAG TTT GAT GTG TGG CAT GGG CA | 8 |
| rTRPM2-3 | 3782 | 3759 | CTC CTC CCT CCT CTC CTT TCT TCC | 9 |
| rTRPM2-4 | 501 | 481 | TTC CCC ACT TTC TGG CTC AG | 10 |
| rTRPM2-5 | 1988 | 1971 | GCT CCC TGT GGT TCT GGA | 11 |
| rTRPM2-6 | 2084 | 2067 | TAT CTT CCT CCT CCT TGG | 12 |
| rTRPM2-7 | 2187 | 2165 | TTC TGG GCT CTT TCC TCA TCC TT | 13 |
| rTRPM2-8 | 4161 | 4139 | CCT CCA CCC TGG TTC CTC TTC CA | 14 |
| rTRPM2-9 | 4270 | 4248 | TAG CAT CTT CCC TGG CTC CCG AG | 15 |

### Example 7

### Reversal

% reversal was calculated according to the following equation:

$$\frac{( Treated - Injured )}{( Baseline - Injured )} \times 100$$

### Example 8

### Distribution of TRPM2 in Human Spinal Cord and DRG

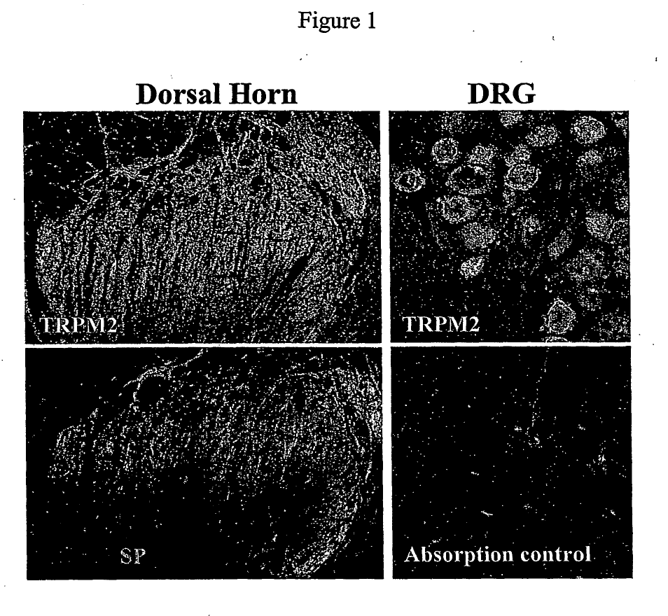

A specific TRPM2 antibody was used to examine the distribution of the channel in human DRG and spinal cord (FIG. 1). In dorsal horn of human spinal cord, TRPM2 immunoreactivity was localized throughout the gray matter. The staining was most intense in inner lamina II and there was no co-localization with SP-immunoreactivity. In DRG, TRPM2 immunoreactivity was present predominantly in large and medium-size neurons. In large neurons, the staining often appeared to decorate the cell membrane. Punctate intracellular staining was also seen in some medium sized neurons. The staining in spinal cord and DRG was abolished in the presence of the peptide antigen (absorption control).

### Example 9

### Distribution of TRPM2 in Rat Spinal Cord, DRG and Sciatic Nerve

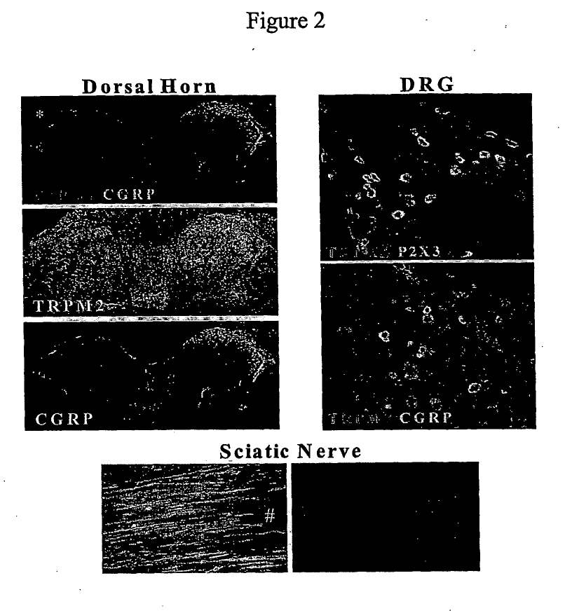

A specific TRPM2 antibody was used to examine the distribution of the channel in rat DRG and spinal cord (FIG. 2). In rat spinal cord, TRPM2 immunoreactivity was localized throughout the gray matter but the staining was most intense in inner lamina II and around the central canal. TRPM2-ir was reduced, but not eliminated after dorsal rhizotomy (indicated by *), suggesting that the staining is present in the central terminals of primary afferent neurons as well as in intrinsic spinal cord neurons. In DRG, TRPM2-ir was present predominantly in large and medium-size neurons. In large neurons, the staining often appeared to decorate the cell membrane. Punctate intracellular staining was also seen in some medium sized neurons. There was limited colocalization with P2X3- and CGRP immunoreactivity.

### Example 10

### Changes in TRPM2 Expression After Spinal Nerve Ligation

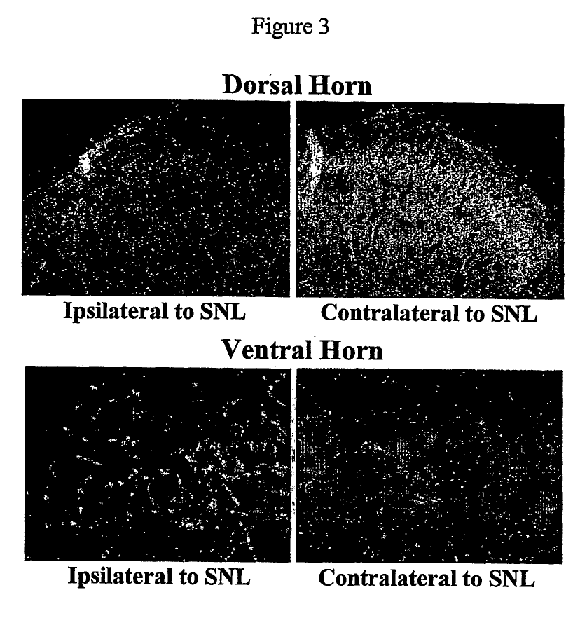

To determine whether TRPM2 expression is regulated under conditions of chronic pain, TRPM2 immunoreactivity was examined in spinal cord of rats with neuropathic pain (SNL model) (FIG. 3). Changes in the amount and distribution of TRPM2 immunoreactivity were observed in both dorsal and ventral horn on the injured side. TRPM2 immunoreactivity was decreased in the dorsal horn ipsilateral to nerve injury. In ventral horn, TRPM2 immunoreactivity appeared to redistribute to the cell membrane and the neuropil of large motor neurons. In addition, in both ventral in dorsal horn, the appearance of small brightly labeled structures was noted, which may represent activated microglia given the localization of TRPM2 in immunocytes.

### Example 11

### Effect of Antisense Oligonucleotides in a Rat Model of Neuropathic Pain

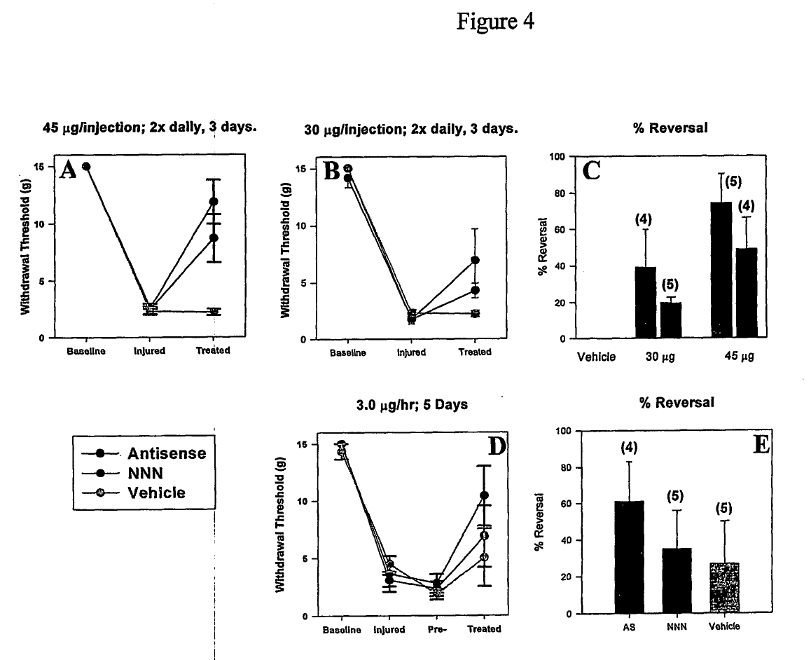

Panels A-C in FIG. 4 are line graphs depicting the effect of TRPM2 antisense oligonucleotides on mechanical pain sensation in rats. Response thresholds to mechanical stimuli were determined after spinal catheterization but before induction of chronic pain (Baseline). Animals were then subjected to a model of chronic neuropathic pain (L5/L6 Spinal Nerve Ligation (SNL)). Nerve injury resulted in decreased response thresholds to mechanical stimuli. Normal rats respond to a noxious heat stimulis applied to their hindpaws with an average latency of 20 seconds (baseline). In animals in which a model of chronic nerve-injury induced (neuropathic) pain has been induced, the response latency decreases to around 10 seconds (injured). This drop is analogous to the abnormal pain sensitivity observed in human patients with chronic nerve injury-related pain (Injured) such as in diabetic neuropathy. Following three days of TRPM2 antisense treatment, there is a significant reduction in the nerve-injury induced hypersensitivity to thermal stimuli (Treated).

Animals were then treated intrathecally with antisense oligonuclebtides directed against rat TRPM2 mRNA twice a day for three days (A=45 μg/injection, B=30 μg/injection) and the effect of treatment on response thresholds was measured. (Treated). The data from panels A and B are depicted in terms of % reversal of injury-induced hypersensitivity in C.

Panels D and E in this example are line graphs depicting the effect of TRPM2 antisense oligonucleotides in a similar experiment. In this case, however, the oligonucleotides were delivered at a rate of 3.0 μg/hour for five days by osmotic minipump following pre-treatment baselines (Pre-) before the effect of treatment was measured (Treated).

The effect of TRPM2 antisense oligonucleotides on mechanical pain sensation can be evaulated. Normal animals rarely respond to stimuli of less than 15 g (baseline). In animals with nerve injury, animals will withdraw from stimuli of only a few grams (Injured). Following three days of TRPM2 antisense treatment, there is a significant reduction in the nerve-injury induced hypersensitivity to mechanical stimuli (Treated).

Animals subjected to inflammation also are significantly more sensitive to thermal and mechanical stimuli, as evidenced by the decreases in their response thresholds compared to pre-inflammation baseline (BL) and uninflamed controls. Following three days of treatment, there is a significant reduction in inflammatory-induced hypersensitivity to both thermal and mechanical stimuli (Treated).

### Example 12

### Quantitative TagMan RT-PCR Analysis of TRPM2 After Antisense Treatment

Quantitative PCR method is used to evaluate TRPM2 mRNA levels in control animals, and in animals with a chronic inflammation in one of the hindpaws treated with a TRPM2 antisense oligonucleotide or with a mismatch oligonucleotide. Treatment with an antisense oligonucleotide reduces the level of TRPM2 mRNA in both inflamed and control animals. TaqMan PCR is carried out using an ABI 7700 sequence detector (Perlin Elmer) on the cDNA samples. TaqMan primer and probe sets are designed from sequences in the GeneBank database using Primer Express (Perkin Elmer).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated antisense oligonucleotide consisting essentially of 10 to 50 nucleotides, wherein said oligonucleotide specifically hybridizes within an accessible region, said region defined by nucleotides 4276 through 4294, 3879 through 3896, 5661 through 5678, or 2821 through 2838 of SEQ ID NO:1, and wherein said oligonucleotide inhibits the production of TRPM2.

2. A composition comprising the isolated antisense oligonucleotide of claim 1.

3. The composition of claim 2, wherein said composition comprises a plurality of isolated antisense oligonucleotides, wherein each antisense oligonucleotide specifically hybridizes within a different accessible region.

4. The isolated antisense oligonucleotide of claim 1, wherein said oligonucleotide comprises a modified backbone.

5. The isolated antisense oligonucleotide of claim 1, wherein said oligonucleotide comprises one or more non-natural internucleoside linkages.

6. The isolated antisense oligonucleotide of claim 1, wherein said oligonucleotide is an oligonucleotide analog.

7. The isolated antisense oligonucleotide of claim 1, wherein said oligonucleotide comprises one or more substituted sugar moieties.

8. The isolated antisense oligonucleotide of claim 1, wherein said oligonucleotide comprises nucleotide base modifications or nucleotide base substitutions.

9. An isolated antisense oligonucleotide consisting essentially of 10 to 50 nucleotides, wherein said oligonucleotide specifically hybridizes within an accessible region, said region defined by nucleotides 273 through 294, 1848 through 1878, 3759 through 3782, 481 through 501, 1971 through 1988, 2067 through 2084, 2165 through 2187, 4139 through 4161, or 4248 through 4270 of SEQ ID NO:2, and wherein said isolated antisense oligonucleotide inhibits the production of TRPM2.

10. A composition comprising the isolated antisense oligonucleotide of claim 9.

11. The composition of claim 10, wherein said composition comprises a plurality of isolated antisense oligonucleotides, wherein each antisense oligonucleotide specifically hybridizes with a different accessible region.

12. An isolated oligonucleotide consisting essentially of the sequence of SEQ ID NO:3, SEQ ID NO:4, SEQ ID NO:5, SEQ ID NO:6, SEQ ID NO:7, SEQ ID NO:8, SEQ ID NO:9; SEQ ID NO:10; SEQ ID NO:11; SEQ ID NO:12; SEQ ID NO:13; SEQ ID NO:14; or SEQ ID NO:15.

13. A method of decreasing production of TRPM2 in cells or tissues, comprising contacting said cells or tissues with an antisense oligonucleotide that specifically hybridizes within an accessible region of TRPM2.

14. The method of claim 13, wherein said contacting step results in an inhibition of pain sensory neurons.

15. A nucleic acid construct comprising a regulatory element operably linked to a nucleic acid encoding a transcript, wherein said transcript specifically hybridizes within one or more accessible regions of TRPM2 mRNA in its native form.

16. A host cell comprising the nucleic acid construct of claim 15.

17. An isolated antisense oligonucleotide that specifically hybridizes within an accessible region of TRPM2 mRNA in its native form, and wherein said antisense oligonucleotide inhibits production of TRPM2.

18. A method for modulating pain in a mammal, said method comprising administering the isolated antisense oligonucleotide of claim 17 to said mammal.

19. A method of identifying a compound that modulates pain in a mammal, the method comprising:
contacting cells comprising a TRPM2 nucleic acid with a compound; and
detecting the amount of TRPM2 RNA or TRPM2 polypeptide in or secreted from said cell,
wherein a difference in the amount of TRPM2 RNA or TRPM2 polypeptide produced in the presence of said compound compared to the amount of TRPM2 RNA or TRPM2 polypeptide produced in the absence of said compound is an indication that said compound modulates pain in said mammal.

20. The method of claim 19, wherein the amount of said TRPM2 RNA is determined by Northern blotting.

21. The method of claim 19, wherein the amount of said TRPM2 polypeptide is determined by Western blotting.

22. The method of claim 19, wherein said compound is an antisense oligonucleotide that specifically hybridizes within an accessible region of TRPM2 mRNA in its native form, wherein said antisense oligonucleotide inhibits production of TRPM2.

23. A method of identifying a compound that modulates pain in a mammal, the method comprising:
contacting cells comprising a TRPM2 nucleic acid with a compound; and
detecting the activity of TRPM2 in or secreted from said cell,
wherein a difference in the activity of TRPM2 in the presence of said compound compared to the activity of TRPM2 in the absence of said compound is an indication that said compound modulates pain in said mammal.

24. A method for modulating pain in a mammal, said method comprising administering a compound to said mammal, wherein said compound modulates the expression of TRPM2.

25. The method of claim 24, wherein said compound is an antisense oligonucleotide that specifically hybridizes within an accessible region of TRPM2 mRNA in its native form, wherein said antisense oligonucleotide inhibits expression of TRPM2.

26. The method of claim 24, wherein said pain is from diabetic neuropathy, gastric pain, postherpetic neuralgia, fibromyalgia, surgery, or chronic back pain.

27. A method for modulating pain in a mammal, said method comprising administering a compound to said mammal, wherein said compound modulates the function of TRPM2.

28. The method of claim 27, wherein said pain is from diabetic neuropathy, gastric pain, postherpetic neuralgia, fibromyalgia, surgery, or chronic back pain.


SEQUENCE LISTING (15 sequences)

SEQ ID NO: 1 - DNA, 6217 bp, Homo sapiens
tgtgcagaattgtacagttgcgaaaccatgtcgctggcagctggtgctggcggtggagacttccctgtgcggtgctcagtgcagctgcacccgtgggggagggagctctttctctggccctgcagtcacctgaggttgttaccattatgaacggccgctgggacccccgcatgtgcatgtactcccccagagtgtccgggggccccagccaagggacacagctcaggcagctgggaacatgtgcaggctgatgaagagaaccggatgagggcttcacatgaggaagcatgtggccaggtcctctcagaacatcagcctcatcttcctgtctctgatctatttcagcaaccaccccatgtgtctctagaaccccagtgtagcgagctggagagaggactgtcctgagggcagcaggcctggttgcagctggcgtgggggtctcagaatggagccctcagccctgaggaaagctggctcggagcaggaggagggctttgaggggctgcccagaagggtcactgacctggggatggtctccaatctccggcgcagcaacagcagcctcttcaagagctggaggctacagtgccccttcggcaacaatgacaagcaagaaagcctcagttcgtggattcctgaaaacatcaagaagaaagaatgcgtgtattttgtggaaagttccaaactgtctgatgctgggaaggtggtgtgtcagtgtggctacacgcatgagcagcacttggaggaggctaccaagccccacaccttccagggcacacagtgggacccaaagaaacatgtccaggagatgccaaccgatgcctttggcgacatcgtcttcacgggcctgagccagaaggtgaaaaagtacgtccgagtctcccaggacacgccctccagcgtgatctaccacctcatgacccagcactgggggctggacgtccccaatctcttgatctcggtgaccgggggggccaagaacttcaacatgaagccgcggctgaagagcattttccgcagaggcctggtcaaggtggctcagaccacaggggcctggatcatcacaggggggtcccacaccggcgtcatgaagcaggtaggcgaggcggtgcgggacttcagcctgagcagcagctacaaggaaggcgagctcatcaccatcggagtcgccacctggggcactgtccaccgccgcgagggcctgatccatcccacgggcagcttccccgccgagtacatactggatgaggatggccaagggaacctgacctgcctagacagcaaccactctcacttcatcctcgtggacgacgggacccacggccagtacggggtggagattcctctgaggaccaggctggagaagttcatatcggagcagaccaaggaaagaggaggtgtggccatcaagatccccatcgtgtgcgtggtgctggagggcggcccgggcacgttgcacaccatcgacaacgccaccaccaacggcaccccctgtgtggttgtggagggctcgggccgcgtggccgacgtcattgcccaggtggccaacctgcctgtctcggacatcactatctccctgatccagcagaaactgagcgtgttcttccaggagatgtttgagaccttcacggaaagcaggattgtcgagtggaccaaaaagatccaagatatcgtccggaggcggcagctgctgactgtcttccgggaaggcaaggatggtcagcaggacgtggatgtggccatcttgcaggccttgctgaaagcctcacggagccaagaccactttggccacgagaactgggaccaccagctgaaactggcagtggcatggaatcgcgtggacattgcccgcagtgagatcttcatggatgagtggcagtggaagccttcagatctgcaccccacgatgacagctgcactcatctccaacaagcctgagtttgtgaagctcttcctggagaacggggtgcagctgaaggagtttgtcacctgggacaccttgctctacctgtacgagaacctggacccctcctgcctgttccacagcaagctgcagaaggtgctggtggaggatcccgagcgcccggcttgcgcgcccgcggcgccccgcctgcagatgcaccacgtggcccaggtgctgcgggagctgctgggggacttcacgcagccgctttatccccggccccggcacaacgaccggctgcggctcctgctgcccgttccccacgtcaagctcaacgtgcagggagtgagcctccggtccctctacaagcgttcctcaggccatgtgaccttcaccatggaccccatccgtgaccttctcatttgggccattgtccagaaccgtcgggagctggcaggaatcatctgggctcagagccaggactgcatcgcagcggccttggcctgcagcaagatcctgaaggaactgtccaaggaggaggaggacacggacagctcggaggagatgctggcgctggcggaggagtatgagcacagagccatcggggtcttcaccgagtgctaccggaaggacgaagagagagcccagaaactgctcacccgcgtgtccgaggcctgggggaagaccacctgcctgcagctcgccctggaggccaaggacatgaagtttgtgtctcacgggggcatccaggccttcctgaccaaggtgtggtggggccagctctccgtggacaatgggctgtggcgtgtgaccctgtgcatgctggccttcccgctgctcctcaccggcctcatctccttcagggagaagaggctgcaggatgtgggcacccccgcggcccgcgcccgtgccttcttcaccgcacccgtggtggtcttccacctgaacatcctctcctacttcgccttcctctgcctgttcgcctacgtgctcatggtggacttccagcctgtgccctcctggtgcgagtgtgccatctacctctggctcttctccttggtgtgcgaggagatgcggcagctcttctatgaccctgacgagtgcgggctgatgaagaaggcagccttgtacttcagtgacttctggaataagctggacgtcggcgcaatcttgctcttcgtggcagggctgacctgcaggctcatcccggcgacgctgtaccccgggcgcgtcatcctctctctggacttcatcctgttctgcctccggctcatgcacatttttaccatcagtaagacgctggggcccaagatcatcattgtgaagcggatgatgaaggacgtcttcttcttcctcttcctgctggctgtgtgggtggtgtccttcggggtggccaagcaggccatcctcatccacaacgagcgccgggtggactggctgttccgaggggccgtctaccactcctacctcaccatcttcgggcagatcccgggctacatcgacggtgtgaacttcaacccggagcactgcagccccaatggcaccgacccctacaagcctaagtgccccgagagcgacgcgacgcagcagaggccggccttccctgagtggctgacggtcctcctactctgcctctacctgctcttcaccaacatcctgctgctcaacctcctcatcgccatgttcaactacaccttccagcaggtgcaggagcacacggaccagatttggaagttccagcgccatgacctgatcgaggagtaccacggccgccccgccgcgccgccccccttcatcctcctcagccacctgcagctcttcatcaagagggtggtcctgaagactccggccaagaggcacaagcagctcaagaacaagctggagaagaacgaggaggcggccctgctatcctgggagatctacctgaaggagaactacctccagaaccgacagttccagcaaaagcagcggcccgagcagaagatcgaggacatcagcaataaggttgacgccatggtggacctgctggacctggacccactgaagaggtcgggctccatggagcagaggttggcctccctggaggagcaggtggcccagacagcccaagccctgcactggatcgtgaggacgctgcgggccagcggcttcagctcggaggcggacgtccccactctggcctcccagaaggccgcggaggagccggatgctgagccgggaggcaggaagaagacggaggagccgggcgacagctaccacgtgaatgcccggcacctcctctaccccaactgccctgtcacgcgcttccccgtgcccaacgagaaggtgccctgggagacggagttcctgatctatgacccacccttttacacggcagagaggaaggacgcggccgccatggaccccatgggagacaccctggagccactgtccacgatccagtacaacgtggtggatggcctgagggaccgccggagcttccacgggccgtacacagtgcaggccgggttgcccctgaaccccatgggccgcacaggactgcgtgggcgcgggagcctcagctgcttcggacccaaccacacgctgtaccccatggtcacgcggtggaggcggaacgaggatggagccatctgcaggaagagcataaagaagatgctggaagtgctggtggtgaagctccctctctccgagcactgggccctgcctgggggctcccgggagccaggggagatgctacctcggaagctgaagcggatcctccggcaggagcactggccgtcttttgaaaacttgctgaagtgcggcatggaggtgtacaaaggctacatggatgacccgaggaacacggacaatgcctggatcgagacggtggccgtcagcgtccacttccaggaccagaatgacgtggagctgaacaggctgaactctaacctgcacgcctgcgactcgggggcctccatccgatggcaggtggtggacaggcgcatcccactctatgcgaaccacaagaccctcctccagaaggcagccgctgagttcggggctcactactgactgtgccctcaggctgggcggctccagtccatagacgttccccccagaaaccagggcttctctctcctgagcctggccaggactcaggctgttcctgggccctgcacatgatggggtttggtggacccagtgcccctcacggctgccgcaagtctgctgcagatgacctcatgaactggaaggggtcaaggtgacccgggaggagagctcaagacagggcacaggctactcagagctgaggggcccctgggacccttggccatcaggcgaggggctgggcctgtgcagctgggcccttggccagagtccactcccttcctggctgtgtcaccccgagcagctcatccaccatggaggtcattggcctgaggcaagttccccggagagtcggggtcccctgtggccccctcaggcctatgtctgtgaggaaggggccctgccactctccccaagagggcctccatgtttcgaggtgcctcaacatggagccttgcctggcctgggctaggggcactgtctgaactcctgactgtcaggataaactccgtgggggtacaggagcccagacaaagcccaggcctgtcaagagacgcagagggcccctgccagggttggccccagggaccctgggacgaggctgcagaagctctccctccctactccctgggagccacgtgctggccatgtggccagggacggcatgagcaggaggcggggacgtgggggccttctggtttggtgtcaacagctcacaggagcgtgaaccatgagggccctcaggaggggaacgtggtaaaacccaagacattaaatctgccatctcaggcctggctggctcttctgtgctttccacaaataaagttcctgacacgtccagggccaggggctgtgtgacggctgcctgaagttctcctcgatcccccggtgagcttcctgcagcctgtggatgtcctgcagcccctcagccctacccccaagtttctcctctgacccatcagctccctgtcttcattttcctaaacctgggctccagcatcgtccccaagcccaccaggccaggatgcaggcatccacatgccctcctccttggcttcccctgcgtggtggtgccaatgtgccctggcacccctgcagaggctccggatggagcctggggctgcctggccactgagcactggccgaggtgatgcccacccttccctggacaggcctctgtcttccacctgacccaaagctctctagccacccccttgtccccag

SEQ ID NO: 2 - DNA, 5448 bp, Rattus norvegicus
ccccaacctcacaaatgagaagagacatccctgaaaggcatctagagatctctctcctctggtggatctggagccgtggaggatggagcccttggaccagagaagaactgactctgatcaagaggagggctttggggtgcagtcccggagggccactgatctgggcatggtccccaatctccgacgaagcaatagcagtctttgcaagagcaggaggcttctgtgctccttcagcagtgagaagcaagaaaaccttagctcatggattcccgagaacatcaagaagaaggaatgtgtgtatttcgtggaaagttccaagctctcggatgcacggaaggtagtgtgtgagtgtggttacacccacgagcagcacattgaagtggccatcaagcctcacaccttccagggcaaggagtgggacccaaagaaacacgtccatgagatgcctacagatgcctttggtgacattgtcttcaccggcctgagccagaaagtggggaagtatgtccgactctcccaggacacgtcgtccattgtcatctaccagcttatgacacagcactggggcctggatgtccccagcctcctcatctctgtgaccggtggggccaagaacttcaacatgaagctgaggttgaagagcatcttccggagaggcctggttaaggtggcccaaaccacgggggcctggatcatcactgggggttcccacaccggtgtgatgaagcaggtgggcgaggcggtacgggacttcagcctaagcagcagctgcaaagaaggcgacgtcatcaccatcggcatagccacgtggggcaccatccacaaccgtgaggcactgatccatcccatgggaggcttccccgctgagtacatgctggatgaggaaggccaagggaacctgacctgcctggacagcaaccactcccacttcatcttggtggatgatgggacccacgggcagtatggtgtggagattccgctgaggactaagctggagaagttcatatcggagcaaacgaaggaaagagggggtgtggccattaagatccccattgtctgcgtggtgttggagggtggccctggcactctgcataccatctacaacgccatcaccaatggcacaccctgcgtgatagtggagggctccggccgagtggctgacgtcatcgctcaggtggccgctctgcccgtctctgagatcaccatctccctgatccagcagaagctcagcgtcttcttccaggagatgtttgagactttcaccgaaaaccagattgtggaatggaccaaaaagatccaagatattgtcaggaggcggcagctgctgacggtcttccgggaaggcaaggatggtcagcaggatgtggatgtcgccattctgcaagctttactgaaagcctctcgaagccaagatcacttcggccacgagaactgggaccatcagctgaagctggccgtggcctggaaccgtgtggacatcgcccgcagtgagatcttcactgatgagtggcagtggaagccttcagacctgcatcccatgatgacagctgccctcatctccaacaagcctgagtttgtgaggctctttctggagaacggggtgcggctgaaggagtttgtcacctgggatactcttctctgcctctacgagaacctggagccatcctgccttttccacagcaagctgcagaaggtgctggcagaagagcatgaacgcttagcctatgcatctgagacaccccggctgcaaatgcaccacgtggcccaggtgctgcgtgagctcctcggagactccacacagctgctgtatccccggccccggtacactgaccggccacggctctcgctgcccatgccacacatcaaactcaacgtgcaaggagtgagcctccggtctctctataagcgatcaacaggccacgttaccttcaccattgacccagtccgcgatcttctcatttgggccatcatccagaaccacagggagctggcgggcatcatctgggctcagagccaggactgcacagcagccgcactggcctgcagcaagatcctgaaggagctgtccaaggaggaggaagatacagacagctctgaggagatgctggcactcgcggatgagtttgagcatagagctatcggtgtcttcaccgagtgctacagaaaggatgaggaaagagcccagaagctgcttgtccgtgtgtctgaggcctgggggaagaccacctgcctgcagctggccctcgaggccaaggacatgaaattcgtgtcacacggagggatccaggctttcctaacgaaggtgtggtggggtcagctctgcgtggacaatggcctgtggaggatcatcctgtgcatgctggccttccctctgctcttcaccggcttcatctccttcagggaaaagaggctgcaggcactgtgccgcccggcccgcgtccgcgccttcttcaacgcgccggtggtcatcttctacctcaatattctctcctactttgccttcctctgcctgtttgcctacgtgctcatggtggacttccagccctcaccatcctggtgcgagtacctcatctacctgtggctcttctccctggtgtgcgaggagacacggcagctattctacgatcccgatggctgcgggctcatgaagatggcgtccctgtacttcagtgacttctggaacaaactggacgttggggccattctgctctttatagcaagactgacctgccgactcatcccagcgacgctgtaccctgggcgcatcatcctgtctttggacttcattatgttctgcctccgcctcatgcacatcttcaccattagcaagacactggggcccaagataatcatcgtgaagcggatgatgaaggacgtcttcttcttcctctttctcctggcggtgtgggtggtgtccttcggagtggccaagcaggccatcctcatccacaatgagagccgcgtggactggatcttccgcggagttatctatcactcttaccttaccatcttcgggcagatcccgacctacattgacggcgtgaatttcagcatggaccagtgcagccccaatggtacagacccctacaagcccaagtgtcctgagagtgactggacagggcaggcacccgccttccccgagtggctgacagtcacccttctctgcctctacctgctcttcgccaacatcctgctgcttaatttgctcatcgccatgttcaactacaccttccagcaggtgcaggagcacacagaccagatctggaagttccagcgccacgacctgattgaggagtaccacggccggcccccggcccctcccccactcatcctcctcagccacctgcagctcctgatcaagaggattgtcctgaagatccccgccaagaggcacaagcagctcaagaacaagctggagaagaatgaggaggcagccctcctgtcctgggagctctacctgaaggagaattacctgcagaaccaacagtaccagcacaaacagcggccagagcagaagatccaggacatcagtgagaaagtggacaccatggtggatctgttggacatggaccgtgtgaagagatcaggctccacagagcagaggctggcctcccttgaggaacaggtgactcagatgggcagatctttgcactggatcgtgacgaccctgaaggacagtggctttggctcaggggccggtgcactgaccctggcagcccaaagggccttcgacgagccagatgctgagctgagtatcaggaagaaaggagaggagggaggagatggctatcatgtgagcgcccggcacctcctctaccctgatgcccgcatcatgcgcttccccgtgcctaatgagaaggtgccttgggaggcagagtttctgatctacgaccctccgttttacacagctgagaagaaggatgcgactctcacagaccctgtgggagacactgcagaacctctgtctaagatcaattacaacgtcgtggacggaccgatggaccgttgcagcttccatgggacctatgtggtccaatatggattccctttgaaccccatgggccgcaccgggttgcgtggtcgtgggagcctcagctggtttggtcccaaccacactctgcagccagttgttacccggtggaagaggaaccagggtggaggcatctgccggaagagtgtcaggaagatgttggaggtgctggtcatgaagttgcctcaatccgagcactgggccttgcctgggggctctcgggagccagggaagatgctaccacggaagctgaaacaggtcctccagcaggagttctgggtgacctttgagaccttgctaaggcaaggaacagaggtgtacaaaggatacgtggatgacccaaggaacacggacaatgcctggatcgagacggtggctgtcagcatccatttccaggaccagaatgatgtggagctgaagaggctggaagagaacctgcaaactcatgatccaaaggagtcggcccgtggcttggagatgtctactgaatggcaggttgtagaccggcggatccctctgtatgtgaaccacaagaagatcctccaaaaggtggcctcgctgtttggcgctcacttctgaccgtgggttcttgtggaagctccaggggaaggggtgatcatccatcaatgacccccctccaagacttggactgggtggcaggttgaggtactgggttggggtggtaggttgtagggctgggttgggtgaccacagggatcttaataagtccccagaggtactgtcctgaaagccacgtctgccacaacaggaggatcacagcatgaggacagaagtgttgtattcagtggctcctgaaatctatgtcctcaagtgccatgccttgattagggtttatggggccctgaccagcacgatgctgccactactcatagacctggttggaagtgctacctctaggacaggtgctttccctcggagggacctctgtcctcatcgtgggagccttctgtcctaggcataaggtagacagaggctatgcctccttcaggaagagacactgctctcataactcctggtcagtgctgagtcacaggcactgaggaaggccgtgagggctttgagctgggatcactgtcatctctagactttagaagggctcctctgaggacactcgggcaggagcacaccctccctcagctccagtgccagctctgctggtcacagaaaacactggaagaggacaacagggtcccatgtcactgcttctagagaaatgtacaactgagaacaataggctttctctcacagggtaggtgacggggtaggtgactcaaggattggtactggctaggagatcaactctgtagtggggggggttgttggaggaacgggcaatgtccgggtttaatgggatggggacaataaagaatgtgtctgatcaaaaaaaaaaaaaaaaa

SEQ ID NO: 3 - DNA, 19 bp, Artificial Sequence
  Oligonucleotide
cgcgtccttcctctctgcc

SEQ ID NO: 4 - DNA, 18 bp, Artificial Sequence
  Oligonucleotide
tgtcctcgatcttctgct

SEQ ID NO: 5 - DNA, 18 bp, Artificial Sequence
  Oligonucleotide
acgtccccgcctcctgct

SEQ ID NO: 6 - DNA, 18 bp, Artificial Sequence
  Oligonucleotide
accaccacgggtgcggtg

SEQ ID NO: 7 - DNA, 22 bp, Artificial Sequence
  Oligonucleotide
cattccttcttcttgatgttct

SEQ ID NO: 8 - DNA, 23 bp, Artificial Sequence
  Oligonucleotide
gagtttgatgtgtggcatgggca

SEQ ID NO: 9 - DNA, 24 bp, Artificial Sequence
  Oligonucleotide
ctcctccctcctctcctttcttcc

SEQ ID NO: 10 - DNA, 20 bp, Artificial Sequence
  Oligonucleotide
ttccccactttctggctcag

SEQ ID NO: 11 - DNA, 18 bp, Artificial Sequence
  Oligonucleotide
gctccctgtggttctgga

SEQ ID NO: 12 - DNA, 18 bp, Artificial Sequence
  Oligonucleotide
tatcttcctcctccttgg

SEQ ID NO: 13 - DNA, 23 bp, Artificial Sequence
  Oligonucleotide
ttctgggctctttcctcatcctt

SEQ ID NO: 14 - DNA, 23 bp, Artificial Sequence
  Oligonucleotide
cctccaccctggttcctcttcca

SEQ ID NO: 15 - DNA, 23 bp, Artificial Sequence
  Oligonucleotide
tagcatcttccctggctcccgag